(19) United States
(12) Patent Application Publication
(10) Pub. No.: US 2023/0210397 A1
(43) Pub. Date: Jul. 6, 2023

(54) NON-INVASIVE AND PERMEABLE RF DIAGNOSIS AND TREATMENT EQUIPMENT AND ITS CATHETER

(71) Applicant: REBECCATECH LLC, Irvine, CA (US)

(72) Inventors: Rongwei Mao, Irvine, CA (US); Yang Zhang, Irvine, CA (US)

(21) Appl. No.: 17/566,777

(22) Filed: Dec. 31, 2021

(51) Int. Cl. A61B 5/05; A61B 18/14; A61B 5/00; A61B 18/00

(52) U.S. Cl. CPC A61B 5/05; A61B 18/1492; A61B 5/6852; A61B 2018/00029; A61B 2562/046; A61B 2018/00142; A61B 2018/1475

(57) ABSTRACT

A non-invasive and permeable RF diagnosis and treatment equipment and its catheter are provided. The catheter which comprises a tube body, a RF electrode array and a flexible protecting net has a retractable cavity, and the RF electrode array is attached to an outer surface of the retractable cavity; the flexible protecting net surrounding outside of the RF electrode array has a connector connected with the tube body and multiple holes. The retractable cavity has a smaller volume contraction state and a larger volume expansion state. Using the catheter, when inserting or pulling out the catheter, the RF electrode array will not contact the inner wall of the organ, but the flexible protecting net contacts the inner wall of the organ. In this way, the scratch of the inner wall of the organ caused by the RF electrode array can be minimized or even avoided through the flexible protecting net.

### FIELD OF THE INVENTION

The present application relates to the field of medical electronic devices, in particular to a device attached to the body surface or cavity for radio frequency treatment, and more specially relates to a non-invasive and permeable RF diagnosis and treatment equipment and its catheter.

### BACKGROUND OF THE INVENTION

Radio frequency treatment has many cutting-edge applications in medicine. RF power is applied through a RF electrode attached to the surface of the tissue. Generally speaking, RF treatment does not require surgery, which is simple and convenient, and the treatment time is short. This method has been widely used in the treatment of kidney and heart, but it is mainly used in the treatment of tumor and cancer. RF treatment is rarely used in large cavities, such as bladder, uterus and so on. Taking the treatment of bladder as an example, the treatment device used must be inserted through a small urethral orifice and then go deep into the bladder. The aperture of the urethra is very narrow, but the volume of the bladder is very large after water filling. The treatment device must accommodate considerable volume changes. However, the tensile deformation of the metal electrode is very limited, it is difficult for the existing RF equipment to be used in organs with huge lumen volume such as bladder, which brings challenges to the development and production of devices.

One of the examples of diagnosis and treatment of large cavity diseases is overactive bladder incontinence (OAB), which will bring great inconvenience to life, especially women aged 45-60. OAB is usually caused by bladder muscle spasm, mainly a problem of bladder nerve and muscle. Detrusor is one of the main muscles of the bladder. When the bladder is full of urine, it contracts to urinate. The contraction and relaxation of detrusor are regulated by the nervous system. OAB is caused by various abnormalities and excessive nerve sensitivity. There are many methods to treat OAB, including behavioral therapy, drug therapy, surgical therapy and RF treatment. Many patients often adopt an attitude of indifference and tolerance, mainly because the existing treatment methods are not ideal. The effect of behavior therapy has a lot to do with whether the patient persists, and it is often ineffective because it is difficult for the patient to persist. Drug therapy, such as anticholinergic drugs, has a certain effect, but is not effective to everyone, and it is often accompanied by great side effects. The method of injecting neurotoxin has a certain effect, but the vast majority of patients will not choose surgical treatment, but still hope to use RF treatment, a non-invasive and safer treatment method. It should be understood that urination or sensing urine pressure is completed through nerve fibers in the inner wall of the bladder. The basic principle of RF treatment of OAB is to reduce the sensitivity of bladder by killing some nerves, so as to normalize the function of bladder. As mentioned above, there is a huge potential market for RF therapy, but there are also great technical obstacles. Only by overcoming the existing technical problems, RF treatment can give full play to its potential value in the treatment of OAB of bladder. A non-invasive and permeable RF diagnosis and treatment equipment generally comprises a RF power supply, an intelligent controller and a catheter. In the process of treatment, the catheter needs to be inserted into the patient's organ, so that the RF electrode array arranged outside the catheter can form good contact with the inner wall of the organ, so that the RF power can smoothly go deep into the deep part of the inner wall of the organ and reach the nerve area, so as to realize the treatment. After the treatment, the catheter can be extracted. The current problem is that during the process of inserting the catheter into the patient's organ and extracting the catheter from the patient's organ, the RF electrode array arranged on the outside of the catheter is easy to scratch the inner wall of the organ.

### SUMMARY OF THE INVENTION

The technical problem to be solved by the application is to provide a catheter for non-invasive permeable RF diagnosis and treatment equipment, which can reduce the risk of scratching the inner wall of organs.

Another technical problem to be solved by the application is to provide a non-invasive and permeable RF diagnosis and treatment equipment with a catheter as described above.

In order to solve the above technical problems, the technical scheme adopted by the application is to provide a catheter used for a non-invasive and permeable RF diagnosis and treatment equipment, wherein, the catheter which comprises a tube body, a RF electrode array and a flexible protecting net has a retractable cavity, and the RF electrode array is attached to an outer surface of the retractable cavity; the flexible protecting net surrounding outside of the RF electrode array has a connector connected with the tube body and multiple holes for RF electrodes of the RF electrode array to protrude or touch the inner wall of the organ, and the retractable cavity has a smaller volume contraction state and a larger volume expansion state; when the retractable cavity expands from a contraction state to an expansion state, the flexible protecting net expands accordingly, and RF electrodes of the RF electrode array are completely or partially aligned with the holes.

By adopting the catheter of the above technical scheme, it can be understood that there is the flexible protecting net between the RF electrode array and the inner wall of the organ during the process of inserting the catheter into the organ or extracting the catheter from the organ when the retractable cavity is in a contraction state. That is, when the catheter is inserted or pulled out, the RF electrode array does not contact the inner wall of the organ, but the flexible protecting net contacts the inner wall of the organ. In this way, the scratch of the RF electrode array on the inner wall of the organ can be minimized or even avoided through the flexible protecting net.

In addition, after the catheter is inserted into the organ, the retractable cavity can be expanded from the contraction state to the expanded state. At this time, the RF electrodes of the RF electrode array are fully or partially aligned with the holes of the net, so that the wavy surface of the inner wall of the organ touches with the RF electrodes of the RF electrode array through the holes of the net. At the same time, under the pressure of the retractable cavity, the RF electrodes can maintain close contact with the tissue surface to form a good electrical contact.

In the catheter provided by the present application, the flexible protecting net is made of elastic material; elastic pressure is always applied to the RF electrode array by the flexible protecting net, keeping the RF electrode array flat on the outer surface of the retractable cavity. In this way, by giving the elastic force to the flexible protecting net, it can be ensured that the RF electrode array can be flattened by the elastic pressure of the flexible protecting net during the retraction of the retractable cavity from the expansion state to the contraction state. The RF electrode array can be flat pasted on the surface of the retractable cavity after repeated expansion and contraction of the retractable cavity. Ensure that when the retractable cavity is in the contraction state, the RF electrode array will not be exposed to the flexible protecting net.

In the catheter provided by the present application, the flexible protecting net comprises a plurality of connecting parts, and two adjacent holes are connected through the connecting part; when the retractable cavity is in the contraction state, the RF electrodes of the RF electrode array are all pressed by the connecting parts. In this way, when the retractable cavity is in the contraction state, the flexible protecting net can apply elastic pressure to the RF electrode of the RF electrode array through the connecting part to further ensure that when the retractable cavity is in the contraction state, the RF electrodes of the RF electrode array are stably housed in the flexible protecting net.

In the catheter provided by the present application, the flexible protecting net is made of one or more materials of siloxane, polyurethane, silica gel, latex, carbon fiber, polyethylene, cross-linked polyethylene, conductive siloxane, polyethylene terephthalate, latex, semi permeable film and nylon. In this way, the flexibility of the flexible protecting net can be ensured, and at the same time, it also has a certain elastic force.

In the catheter provided by the present application, the flexible protecting net comprises a plurality of sequentially connected segments with different materials. The elastic force of different segments of the flexible protecting net is controlled through the selection of materials. Here, the elastic force of the segment corresponding to the RF electrode array is designed to be large, so as to further ensure that the RF electrode array is always attached to the outer surface of the retractable cavity.

In the catheter provided by the present application, the flexible protecting net comprises a relative front end and a back end, the front end and the back end of the flexible protecting net are respectively provided with the connectors, the two connectors are respectively connected with the retractable cavity of the tube body.

In the catheter provided by the present application, only a front end of the flexible protecting net is provided with the connector, the connector of the front end of the flexible protecting net is tightly enclosed with a front end of the tube body, a back end of the flexible protecting net is coated with a back end of the tube body. In this way, when the back end of the catheter is inserted into the urethral orifice, the flexible protecting net will not be rubbed off due to physical friction.

In the catheter provided by the present application, the size of the holes of the flexible protecting net is uneven. Here, the size of the holes at the back end of the flexible protecting net is small and the size of the holes at the front end is large. It can be understood that the density of the holes at the back end of the flexible protecting net is greater than that at the front end of the flexible protecting net. That is, the density of the holes at the back end of the flexible protecting net is designed to be dense to ensure that the back end of the flexible protecting net can cover the back end of the tube body. The density of the holes at the front end of the flexible protecting net is designed to be sparse to ensure that the RF electrode can contact the tissue surface in the expansion state.

In the catheter provided by the present application, thickness of different areas of the flexible protecting net is different. Here, the thickness of the back end of the flexible protecting net is designed to be thick to ensure that the back end of the flexible protecting net has sufficient strength and prevent the back end of the flexible protecting net from being pierced by the back end of the tube body. At the same time, the thickness of the front end of the flexible protecting net is designed to be thin to further ensure that the RF electrode can contact the tissue surface in the expansion state.

In the catheter provided by the present application, surface of the flexible protecting net or the RF electrode array or the retractable cavity is coated with a coating lubricant, and the coating lubricant is dry or liquid. In this way, the process of inserting or extracting the catheter into or out of the organ can be smoother.

In the catheter provided by the present application, a fixed structure is provided at a position where the retractable cavity is connected with the flexible protecting net. In some embodiments, the fixed structure comprises a raised ring protruding on the surface of the retractable cavity. In other embodiments, the fixed structure comprises an annular limiting groove recessed on the surface of the retractable cavity. It should be understood that the fixed structure can effectively prevent the connector from sliding on the surface of the tube body.

In the catheter provided by the present application, the RF electrode array is supported by a flexible film, one end of the flexible film is fixed on an outer surface of the retractable cavity and the other end is not fixed. It can be understood that since the other end of the flexible film at the bottom of the RF electrode array is not connected with the outer surface of the retractable cavity, the RF electrode array can be attached to the outer surface of the retractable cavity with the expansion of the retractable cavity without being broken, The RF electrode array can adapt to the huge volume change of the retractable cavity.

In the catheter provided by the present application, the two ends of the catheter are respectively provided with a first body fluid input and output port and a second body fluid input and output port communicated with each other; the retractable cavity is arranged between the first body fluid input and output port and the second body fluid input and output port and closer to the second body fluid input and output port; one end close to the first body fluid input and output port is connected with a refrigerant injection port passing through the catheter and connected with the retractable cavity, and the retractable cavity is filled with refrigerant. In this way, the refrigerant can be injected or extracted into the retractable cavity through the refrigerant injection port, so as to realize the expansion and contraction of the retractable cavity. In addition, the RF electrode array in contact with the retractable cavity can always maintain a low temperature and effectively protect the mucosal tissue on the inner wall surface of the organ from burning.

In the catheter provided by the present application, when the retractable cavity expands from the contraction state to the expansion state, a size of the hole is larger than that of the RF electrode, and some RF electrodes of the RF electrode array penetrate from the holes. In this way, the contact area between the RF electrode and the tissue surface is increased as much as possible, and the stability and reliability of the electrical contact between the RF electrode and the tissue surface are improved.

In the catheter provided by the present application, the connector is connected with the retractable cavity of the tube body. In this way, the flexible protecting net only covers the retractable cavity of the tube body, which can ensure that the flexible protecting net completely surrounds the RF electrode array and reduce the material consumption of the flexible protecting net. Most importantly, when the retractable cavity is in the expansion state, the flexible protecting net can better fully contact the RF electrode array, which is more favorable for the flexible protecting net to apply elastic pressure to the whole RF electrode array.

In order to solve the other technical problem, the technical scheme adopted by the invention is to provide a non-invasive and permeable RF diagnosis and treatment equipment, which comprises a RF power supply, an intelligent controller and the above catheter, the RF power supply is electrically connected with the RF electrode array through the intelligent controller.

When the non-invasive permeable RF diagnosis and treatment equipment of the above technical scheme is used for treatment, after the catheter is inserted into the organ when the retractable cavity is in the contraction state, refrigerant is injected into the retractable cavity through the refrigerant injection port to expand the retractable cavity to the expanded state; at this time, the RF electrode of the RF electrode array is in contact with the inner wall of the organ; then, turn on the RF power supply, and the intelligent controller automatically controls the parameters of RF treatment (including RF power, treatment time, pulse cycle and width) to enable the RF electrode array to treat organs; after the treatment is completed, the refrigerant in the retractable cavity is extracted from the refrigerant injection port to retract the retractable cavity to the contraction state, and then the catheter is extracted from the organ. So far, the intelligent RF treatment of organs has been realized and the best treatment effect has been obtained.

In the non-invasive and permeable RF diagnosis and treatment equipment provided by the present application, further comprises a signal detector and a signal source; the signal detector is arranged outside the organ when using the diagnosis and treatment equipment, and the catheter is integrated with a RF electrode array or a signal transmitter; the signal detector is used to detect a signal sent by the signal transmitter and locate or diagnose a lesion in three dimensions according to the signal. In this way, the positioning treatment is realized through the signal transmitter and the signal detector.

The following beneficial effects can be achieved by the present application:

1. there is the flexible protecting net between the RF electrode array and the inner wall of the organ during the process of inserting the catheter into the organ or extracting the catheter from the organ when the retractable cavity is in a contraction state. That is, when the catheter is inserted or pulled out, the RF electrode array does not contact the inner wall of the organ, but the flexible protecting net contacts the inner wall of the organ. In this way, the scratch of the RF electrode array on the inner wall of the organ can be minimized or even avoided through the flexible protecting net;

2. by giving the elastic force to the flexible protecting net, it can be ensured that the RF electrode array can be flattened by the elastic pressure of the flexible protecting net during the retraction of the retractable cavity from the expansion state to the contraction state. The RF electrode array can be flat pasted on the surface of the retractable cavity after repeated expansion and contraction of the retractable cavity. Ensure that when the retractable cavity is in the contraction state, the RF electrode array will not be exposed to the flexible protecting net;

when the retractable cavity is in the contraction state, the flexible protecting net can apply elastic pressure to the RF electrode of the RF electrode array through the connecting part to further ensure that when the retractable cavity is in the contraction state, the RF electrodes of the RF electrode array are stably housed in the flexible protecting net.

### BRIEF DESCRIPTION OF THE DRAWINGS

In order to more clearly explain the technical scheme in the embodiment of the application, the attached drawings needed to be used in the description of the embodiment will be briefly introduced. Obviously, the drawings described below are only some embodiments of the application. For ordinary technicians in the art, without paying creative labor, other drawings may also be obtained from these drawings:

FIG. 1 is a structural diagram of the non-invasive and permeable RF diagnosis and treatment equipment provided by the first embodiment of the present application;

FIG. 2 is a structural diagram of the catheter provided by the first embodiment of the present application when the retractable cavity is in an expansion state;

FIG. 3 is a structural diagram of the catheter provided by the first embodiment of the present application when the retractable cavity is in a contraction state;

FIG. 4 is a partial structural diagram of the flexible protecting net provided by the first embodiment of the present application;

FIG. 5 is a structural diagram of the catheter provided by the second embodiment of the present application when the retractable cavity is in an expansion state.

wherein, the meanings represented by the numbers in the drawings are as follows:

| | | | |
|---|---|---|---|
| catheter | 1 | RF power supply | 2 |
| intelligent controller | 3 | tube body | 11 |
| RF electrode array | 12 | flexible protecting net | 13 |
| first body fluid input and | 111 | second body fluid input | 112 |
| output port | | and output port | |
| refrigerant injection port | 113 | retractable cavity | 114 |
| connector | 131 | hole | 132 |
| connecting part | 133 | | |

### DETAILED DESCRIPTION OF THE PREFERRED EMBODIMENT

In order to facilitate the understanding of the invention, the invention will be described more comprehensively below with reference to the relevant drawings. Typical embodiments of the invention are given in the accompanying drawings. However, the invention can be implemented in many different forms and is not limited to the embodiments described herein. On the contrary, the purpose of providing these embodiments is to make the disclosure of the invention more thorough and comprehensive.

Unless otherwise defined, all technical and scientific terms used herein have the same meanings as those commonly understood by those skilled in the technical field of the invention. The terms used in the description of the invention herein are only for the purpose of describing specific embodiments and are not intended to limit the invention.

### The First Embodiment

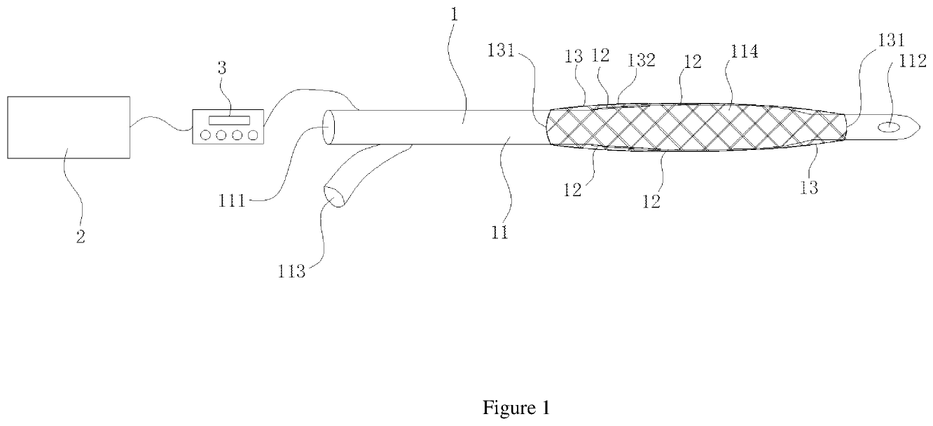

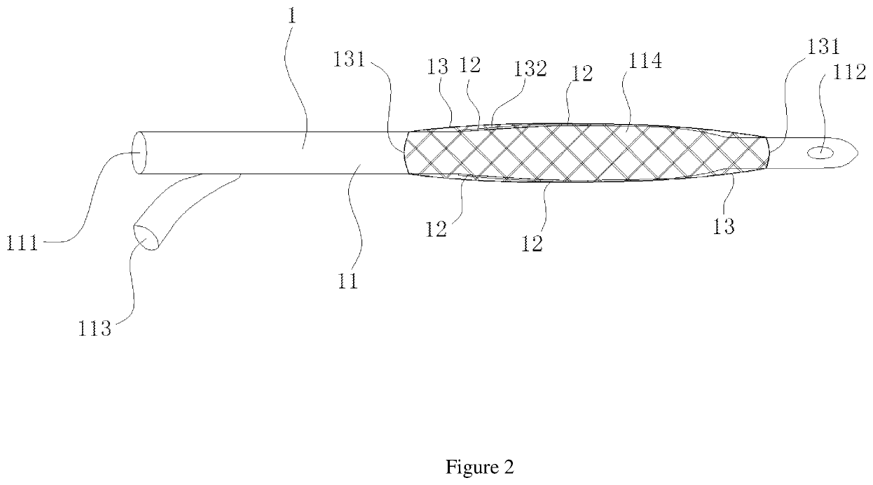

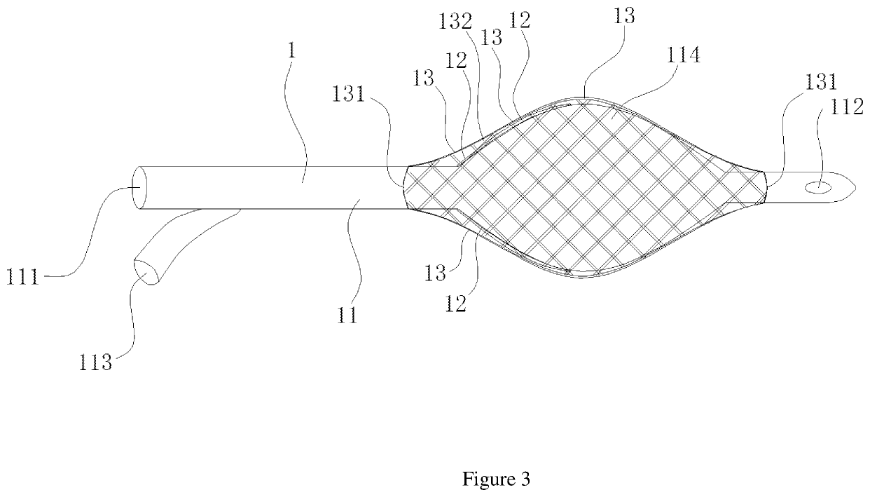

This embodiment provides a non-invasive and permeable RF diagnosis and treatment equipment. Referring to FIG. 1, which provides a structural diagram of the non-invasive and permeable RF diagnosis and treatment equipment according to this embodiment. As shown in FIG. 1, the non-invasive and permeable RF diagnosis and treatment equipment comprises a RF power supply 2, an intelligent controller 3 and a catheter 1. The RF power supply 2 is electrically connected with the RF electrode array 12 through the intelligent controller 3. The catheter 1 comprises a tube body 11, a RF electrode array 12 and a flexible protecting net 13. As can be seen in FIG. 1, the two ends of the catheter 1 are respectively provided with a first body fluid input and output port 111 and a second body fluid input and output port 112 communicated with each other; the retractable cavity 114 is arranged between the first body fluid input and output port 111 and the second body fluid input and output port 112 and closer to the second body fluid input and output port 112; one end close to the first body fluid input and output port 111 is connected with a refrigerant injection port 113 passing through the catheter 1 and connected with the retractable cavity 114, and the retractable cavity 114 is filled with refrigerant. In this way, the refrigerant can be injected or extracted into the retractable cavity 114 through the refrigerant injection port 113, so as to realize the expansion and contraction of the retractable cavity 114. In addition, the RF electrode array 12 in contact with the retractable cavity 114 can always maintain a low temperature and effectively protect the mucosal tissue on the inner wall surface of the organ from burning. The RF electrode array 12 is attached to an outer surface of the retractable cavity 114, and the flexible protecting net 13 surrounding outside of the RF electrode array 12 comprises two connectors 131 and a plurality of holes 132. The two connectors 131 are respectively connected with the tube body 11. The net holes 132 can be used for the RF electrodes of the RF electrode array 12 to contact with the inner wall of the organ. And the retractable cavity 114 has a contraction state and an expansion state. Referring to FIG. 2, which is a structural diagram of the catheter provided by the first embodiment of the present application when the retractable cavity is in an expansion state. As shown in FIG. 2, when the retractable cavity 114 expands from the contraction state to the expansion state, the flexible protecting net 13 expands, the RF electrodes of the RF electrode array 12 are exposed from the holes 132, and the inner wall of the organ is in contact with the RF electrodes. It should be noted that when the size of the holes is larger than the size of the RF electrode, the RF electrode of the RF electrode array 12 can penetrate out of the flexible protecting net 13 from the holes 132. Referring to FIG. 3, which is a structural diagram of the catheter provided by the first embodiment of the present application when the retractable cavity is in a contraction state. As shown in FIG. 3, when the retractable cavity 114 shrinks from the expansion state to the contraction state, the flexible protecting net 13 shrinks, and the RF electrode of the RF electrode array 12 is still wrapped by the flexible protecting net 13; it should be noted that when the size of the hole is larger than the size of the RF electrode, the RF electrode of the RF electrode array 12 returns from the hole 132 to the flexible protecting net 13. In this embodiment, the intelligent controller 3 includes an information display unit, a logic control unit, a communication module, and a RF controller. For the specific structure and working principle of the intelligent controller 3, please refer to the intelligent controller 3 disclosed in the Chinese patent No. 201610753065.2.

When the non-invasive permeable RF diagnosis and treatment equipment of the above technical scheme is used for treatment, after the catheter 1 is inserted into the organ when the retractable cavity 114 is in the contraction state, refrigerant is injected into the retractable cavity 114 through the refrigerant injection port 113 to expand the retractable cavity 114 to the expanded state; at this time, the RF electrode of the RF electrode array 12 is in contact with the inner wall of the organ; then, turn on the RF power supply 2, and the intelligent controller 3 automatically controls the parameters of RF treatment (including RF power, treatment time, pulse cycle and width) to enable the RF electrode array 12 to treat organs; after the treatment is completed, the refrigerant in the retractable cavity 114 is extracted from the refrigerant injection port 113 to retract the retractable cavity 114 to the contraction state, and then the catheter 1 is extracted from the organ. So far, the intelligent RF treatment of organs has been realized and the best treatment effect has been obtained.

It can be understood that there is the flexible protecting net 13 between the RF electrode array 12 and the inner wall of the organ during the process of inserting the catheter 1 into the organ or extracting the catheter 1 from the organ when the retractable cavity 114 is in a contraction state. That is, when the catheter 1 is inserted or pulled out, the RF electrode array 12 does not contact the inner wall of the organ, but the flexible protecting net 13 contacts the inner wall of the organ. In this way, the scratch of the RF electrode array 12 on the inner wall of the organ can be minimized or even avoided through the flexible protecting net 13.

In addition, after the catheter 1 is inserted into the organ, the retractable cavity 114 can be expanded from the contraction state to the expanded state. At this time, the RF electrodes of the RF electrode array 12 are fully or partially aligned with the holes 132, so that the wavy surface of the inner wall of the organ contact with the RF electrodes of the RF electrode array 12 through the holes 132. At the same time, under the pressure of the retractable cavity 114, the RF electrodes can maintain close contact with the tissue surface to form a good electrical contact.

It should be understood that the RF electrode array 12 may be attached to the front and/or back ends of the outer surface of the retractable cavity 114.

Further, the flexible protecting net comprises a plurality of sequentially connected segments with different materials. The elastic force of different segments of the flexible protecting net is controlled through the selection of materials. Here, the elastic force of the segment corresponding to the RF electrode array is designed to be large, so as to further ensure that the RF electrode array is always attached to the outer surface of the retractable cavity.

Further, when the retractable cavity expands from the contraction state to the expansion state, the size of the hole is larger than the size of the RF electrode, and part of the RF electrode array passes through the hole. Specifically, the position and size of the hole 132 can be designed according to different materials, wherein the position can be matched with the position of the electrode array 12 to ensure that the RF electrode of the electrode RF electrode array 12 can be directly exposed through the hole 132 when the flexible protecting net is extended. Preferably, when the flexible protecting net is stretched, the size of the hole 132 is larger than that of the RF electrode, so that the RF electrode can be completely exposed through one of the holes 132, so that the contact area between the RF electrode and the tissue surface can be increased as much as possible, improving the stability and reliability of the electrical contact between the RF electrode and the tissue surface.

Further, the connection between the connector 131 at the front end of the flexible protecting net and the retractable cavity 114 and the connection between the connector 131 at the back end of the flexible protecting net and the retractable cavity 114 can use the material with better fastening effect and/or the hole 132 with relatively small size to prevent the flexible protecting net from falling off or curling.

Further, the thickness of the flexible protecting net is uneven. Because the thickness of the hole 132 is different in some places where the hole 132 is opened, there will be an estimate where the hole 132 is opened to ensure that the RF electrode of the electrode RF electrode array 12 can be directly exposed.

Further, the surface of the flexible protecting net 13 or the electrode RF electrode array 12 or the retractable cavity 114 is coated with a water-soluble coating lubricant (e.g., n-dodecyl-beta-d-maltoside|dodecyl)-β-D-maltoside, GDN sugar diosgenin, etc.), the coating lubricant is dry in the air, but becomes smooth in the presence of water, so as to facilitate the insertion of the catheter into the balloon and smooth opening. Of course, in some other embodiments, a liquid coating lubricant (e.g., sodium hyaluronate, dilute glycerol, etc.) can also be coated on the surface of the flexible protecting net 13 or the electrode RF electrode array 12 or the retractable cavity 114.

Further, the flexible protecting net 13 is longer than the retractable cavity 114 (at contraction state) in length such that the flexible protecting net 13 is covering the whole surface of the retractable cavity 114. The flexible protecting net 13 is fixed on the tube body 11. In this embodiment, the fixed structure can be a raised ring protruding on the surface of the tube body or the retractable cavity 114, and the connector of the flexible protecting net is tightly enclosed with the raised ring. In some other embodiments, the fixed structure may be an annular limiting groove concave on the surface of the tube body or the retractable cavity.

Further, in this embodiment, the flexible protecting net 13 is made of elastic material, and the flexible protecting net 13 always applies elastic pressure to the RF electrode array 12 so that the RF electrode array 12 is always flat on the outer surface of the retractable cavity 114. Here, it is preferable that the flexible protecting net 13 is made of siloxane, so that the flexible protecting net 13 can have certain flexibility and elastic force. It should be noted that by giving elastic force to the flexible protecting net 13, it can be ensured that the RF electrode array 12 can be flattened by the elastic pressure of the flexible protecting net 13 during the retraction of the retractable cavity 114 from the expansion state to the contraction state. After the retractable cavity 114 expands and contracts repeatedly, the RF electrode array 12 can be flat attached to the surface of the retractable cavity 114. Ensure that when the retractable cavity 114 is in the contraction state, the RF electrode array 12 is fully covered by the flexible protecting net 13, and further ensure that the RF electrode array 12 will not scratch the inner wall of the organ during the extraction of the catheter 1.

Of course, in some other embodiments, the flexible protecting net 13 can also be made of one or more materials of siloxane, polyurethane, silica gel, latex, carbon fiber, polyethylene, cross-linked polyethylene, polyethylene terephthalate, semi permeable film and polyamide.

Of course, in some other embodiments, the flexible protecting net 13 can also be made of non elastic material. The flexible protecting net 13 will collapse/shrink naturally without the support of the support structure, or the protruding RF electrode array 12 can be flattened to a certain extent and pasted on the wall of the catheter 1.

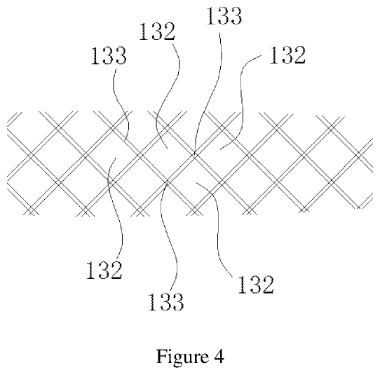

Further, in this embodiment, referring to FIG. 4, which is a partial structural diagram of the flexible protecting net provided by the first embodiment of the present application. As shown in FIG. 4, the flexible protecting net 13 comprises a plurality of connecting parts 133, and two adjacent holes 132 are connected through the connecting part 133; when the retractable cavity 114 is in the contraction state, the RF electrodes of the RF electrode array 12 are all pressed by the connecting parts 133. In this way, when the retractable cavity 114 is in the contraction state, the flexible protecting net 13 can apply elastic pressure to the RF electrode of the RF electrode array 12 through the connecting part 133 to further ensure that when the retractable cavity 114 is in the contraction state, the RF electrodes of the RF electrode array 12 are stably housed in the flexible protecting net 13.

Further, in this embodiment, the RF electrode array 12 is supported by a flexible film, one end of the flexible film is fixed on the tube body 11 and the other end is not fixed. It can be understood that since the other end of the flexible film at the bottom of the RF electrode array 12 is not connected with the outer surface of the retractable cavity 114, the RF electrode array 12 can be attached to the outer surface of the retractable cavity 114 with the expansion of the retractable cavity 114 without being broken, The RF electrode array 12 can adapt to the huge volume change of the retractable cavity 114.

Further, when the retractable cavity expands from the contraction state to the expansion state, a size of the hole is larger than that of the RF electrode, and some RF electrodes of the RF electrode array penetrate from the holes. In this way, the contact area between the RF electrode and the tissue surface is increased as much as possible, and the stability and reliability of the electrical contact between the RF electrode and the tissue surface are improved.

### The Second Embodiment

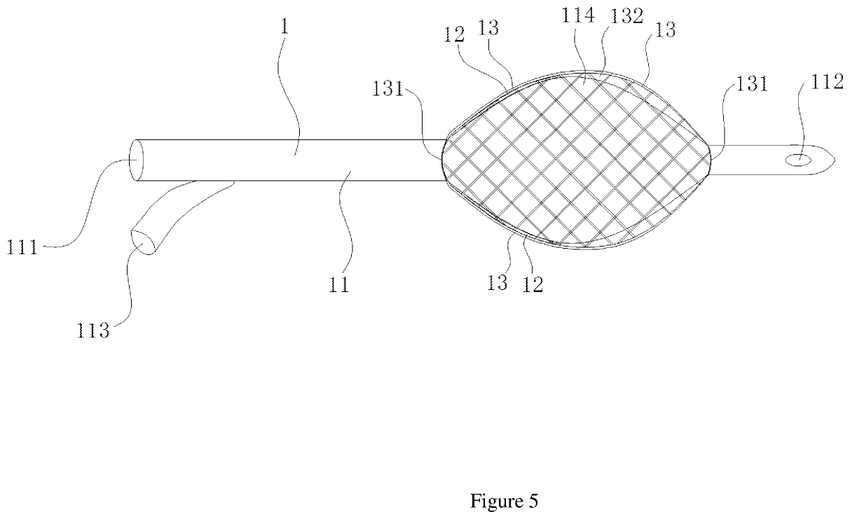

This embodiment provides a non-invasive and permeable RF diagnosis and treatment equipment, the difference between the non-invasive and permeable RF diagnosis and treatment equipment provided by this embodiment and the first embodiment is that the structure of catheter 1 is different. Referring to FIG. 5, which is a structural diagram of the catheter 1 provided by the second embodiment of the present application when the retractable cavity 114 is in an expansion state. As shown in FIG. 5, the two connectors 131 are respectively connected with the retractable cavity 114 of the tube body 11, that is, the flexible protecting net 13 only covers the retractable cavity 114 of the tube body 11. In this way, it can be ensured that the flexible protecting net 13 completely surrounds the RF electrode array 12, and the material consumption of the flexible protecting net 13 can be reduced. More importantly, compared with FIG. 2 and FIG. 4, it can be found that when the retractable cavity 114 is in the expanded state, the flexible protecting net 13 in this embodiment can be in better overall contact with the end of the RF electrode array 12 fixed to the retractable cavity 114 than in the first embodiment. It is more advantageous for the flexible protecting net 13 to apply elastic pressure to the whole RF electrode array 12.

### The Third Embodiment

This embodiment provides a non-invasive and permeable RF diagnosis and treatment equipment, the difference between the non-invasive and permeable RF diagnosis and treatment equipment provided by this embodiment and the first embodiment is that, the non-invasive and permeable RF diagnosis and treatment equipment provided by this embodiment further comprises a signal detector and a signal source; when using the diagnosis and treatment equipment, the signal detector is arranged outside the organ, and the catheter 1 is integrated with a RF electrode array 12 or a signal transmitter. The signal detector is used to detect a signal sent by the signal transmitter and locate or diagnose a lesion in three dimensions according to the signal. In this way, the positioning treatment is realized through the signal transmitter and the signal detector. Here, the RF electrode array 12 and the signal transmitter are separate structures or integrated structures, and the signal transmitter and the signal source are separate structures or integrated structures. Specifically, the RF electrode integrated in the catheter 1 can be used as a signal transmitter. Of course, an additional signal transmitter can be integrated on the flexible film. The signal intensity received by the signal detector located outside the body is related to the distance and angle between them. In addition, the diseased tissue will be different from the normal tissue. Using this principle, we can use it to locate the RF electrode and diagnose tissue lesions.

### The Fourth Embodiment

This embodiment provides a non-invasive and permeable RF diagnosis and treatment equipment, the difference between the non-invasive and permeable RF diagnosis and treatment equipment provided by this embodiment and the first embodiment is that, only a front end of the flexible protecting net is provided with the connector, the connector of the front end of the flexible protecting net is tightly enclosed with a front end of the tube body, a back end of the flexible protecting net is coated with a back end of the tube body. That is, compared with the first embodiment, only the front end of the flexible protecting net is provided with the connector, while the back end is not designed with the connector, but directly coated on the periphery of the back end of the tube body (i.e. the end where the second body fluid input and output port 112 is located). In this way, when the back end of the catheter is inserted into the urethral orifice, the flexible protecting net will not be rubbed off due to physical friction.

Further, the flexible protecting net comprises an opposite front end and a back end. The flexible protecting net is divided into a plurality of successively connected segments from front to back. The material of each segment is different. The elastic force of different segments of the flexible protecting net is controlled through the selection of material. Here, the elastic force of the segment corresponding to the RF electrode array is designed to be large, which is further ensured that the RF electrode array is always attached to the outer surface of the retractable cavity.

Further, the size of the holes of the flexible protecting net is uneven. Here, the size of the holes at the back end of the flexible protecting net is small and the size of the holes at the front end is large. It can be understood that the density of the holes at the back end of the flexible protecting net is greater than that at the front end of the flexible protecting net. That is, the density of the holes at the back end of the flexible protecting net is designed to be dense to ensure that the back end of the flexible protecting net can cover the back end of the tube body. The density of the holes at the front end of the flexible protecting net is designed to be sparse to ensure that the RF electrode can contact the tissue surface in the expansion state.

Further, thickness of different areas of the flexible protecting net is different. Here, the thickness of the back end of the flexible protecting net is designed to be thick to ensure that the back end of the flexible protecting net has sufficient strength and prevent the back end of the flexible protecting net from being pierced by the back end of the tube body. At the same time, the thickness of the front end of the flexible protecting net is designed to be thin to further ensure that the RF electrode can contact the tissue surface in the expansion state.

The above illustration describes the implementation of inventions, but inventions are not limited to the above specific implementation methods. The above specific implementation methods are only schematic rather than restrictive. Under the inspiration of inventions, ordinary technicians in this field can also make many forms without breaking away from the scope protected by invention purposes and rights requirements, which are within the protection of inventions.

1. A catheter used for a non-invasive and permeable RF diagnosis and treatment equipment, wherein, the catheter (1) which comprises a tube body (11), a RF electrode array (12) and a flexible protecting net (13) has a retractable cavity (114), and the RF electrode array (12) is attached to an outer surface of the retractable cavity (114); the flexible protecting net (13) surrounding outside of the RF electrode array (12) has a connector (131) connected with the tube body (11) and multiple holes (132); when the retractable cavity (114) expands from a contraction state to an expansion state, the flexible protecting net (13) expands accordingly, and RF electrodes of the RF electrode array (12) are completely or partially aligned with the holes (132).

2. The catheter according to claim 1, wherein, the flexible protecting net (13) is made of elastic material; elastic pressure is always applied to the RF electrode array (12) by the flexible protecting net (13), keeping the RF electrode array (12) flat on the outer surface of the retractable cavity (114).

3. The catheter according to claim 2, wherein, the flexible protecting net (13) comprises a plurality of connecting parts (133), and two adjacent holes (132) are connected through the connecting part (133); when the retractable cavity (114) is in the contraction state, the RF electrodes of the RF electrode array (12) are all pressed by the connecting parts (133).

4. The catheter according to claim 2, wherein, the flexible protecting net (13) is made of one or more materials of siloxane, polyurethane, silica gel, latex, carbon fiber, polyethylene, cross-linked polyethylene, conductive siloxane, polyethylene terephthalate, semi permeable film and polyamide.

5. The catheter according to claim 4, wherein, the flexible protecting net (13) comprises a plurality of sequentially connected segments with different materials.

6. The catheter according to claim 2, wherein, the size of the holes (132) of the flexible protecting net (13) is uneven.

7. The catheter according to claim 2, wherein, thickness of different areas of the flexible protecting net (13) is different.

8. The catheter according to claim 1, wherein, the flexible protecting net (13) comprises a relative front end and a back end, the front end and the back end of the flexible protecting net are respectively provided with the connectors, the two connectors (131) are respectively connected with the retractable cavity (114) of the tube body (11).

9. The catheter according to claim 1, wherein, only a front end of the flexible protecting net (13) is provided with the connector, the connector of the front end of the flexible protecting net is tightly enclosed with a front end of the tube body, a back end of the flexible protecting net is coated with a back end of the tube body.

10. The catheter according to claim 1, wherein, surface of the flexible protecting net (13) or the RF electrode array (12) or the retractable cavity (114) is coated with a coating lubricant.

11. The catheter according to claim 10, wherein, the coating lubricant is dry or liquid.

12. The catheter according to claim 1, wherein, a fixed structure is provided at a position where the retractable cavity (114) is connected with the flexible protecting net (13).

13. The catheter according to claim 12, wherein, the fixed structure comprises a raised ring protruding on the surface of the retractable cavity (114).

14. The catheter according to claim 12, wherein, the fixed structure comprises an annular limiting groove recessed on the surface of the retractable cavity (114).

15. The catheter according to claim 1, wherein, the RF electrode array (12) is supported by a flexible film, one end of the flexible film is fixed on an outer surface of the retractable cavity (114) and the other end is not fixed.

16. The catheter according to claim 1, wherein, the two ends of the catheter (1) are respectively provided with a first body fluid input and output port (111) and a second body fluid input and output port (112) communicated with each other; the retractable cavity (114) is arranged between the first body fluid input and output port (111) and the second body fluid input and output port (112) and closer to the second body fluid input and output port (112); one end close to the first body fluid input and output port (111) is connected with a refrigerant injection port (113) passing through the catheter (1) and connected with the retractable cavity (114), and the retractable cavity (114) is filled with refrigerant.

17. The catheter according to claim 1, wherein, when the retractable cavity (114) expands from the contraction state to the expansion state, a size of the hole is larger than that of the RF electrode, and some RF electrodes of the RF electrode array (12) penetrate from the holes (132).

18. The catheter according to claim 1, wherein, the connector (131) is connected with the retractable cavity of the tube body (11).

19. A non-invasive and permeable RF diagnosis and treatment equipment, wherein, comprises a RF power supply (2), an intelligent controller (3) and a catheter according to claim 1, the RF power supply (2) is electrically connected with the RF electrode array through the intelligent controller (3).

20. The non-invasive and permeable RF diagnosis and treatment equipment according to claim 19, wherein, further comprises a signal detector and a signal source; the signal detector is arranged outside the organ when using the diagnosis and treatment equipment, and the catheter (1) is integrated with a RF electrode array (12) or a signal transmitter; the signal detector is used to detect a signal sent by the signal transmitter and locate or diagnose a lesion in three dimensions according to the signal.
